(19) United States
(12) Patent Application Publication
(10) Pub. No.: US 2009/0176697 A1
(43) Pub. Date: Jul. 9, 2009

(54) Method to obtain the human Chorionic Gonadotropin (hCG)/cyclodextrin complex for oral administration, product obtained by this method and clinical and therapeutic use of the complex human Chorionic Gonadotropin (hCG)/cyclodextrin

(75) Inventors: Daniel Oscar Belluscio, Capital Federal (AR); Sergio Ariel Vaney, Capital Federal (AR)

Correspondence Address: BELLUSCIO, DANIEL OSCAR, ESTADOS UNIDOS 972 10 E, CAPITAL FEDERAL / BUENOS AIRES, 1101, AR

(73) Assignees: Dr. Daniel Oscar Belluscio, Capital Federal (AR); Dr. Sergio Ariel Vaney, Capital Federal (AR)

(21) Appl. No.: 12/318,073

(22) Filed: Dec. 22, 2008

(30) Foreign Application Priority Data
Jan. 8, 2008 (AR) P20080100070

(51) Int. Cl. A61K 38/24; A61P 3/04; A61P 3/10

(52) U.S. Cl. 514/8

(57) ABSTRACT

To inform about the clinical utility human Chorionic Gonadotropin (hCG) complexed with cyclodextrins for oral administration, its utility in different pathologies and method for obtaining it.

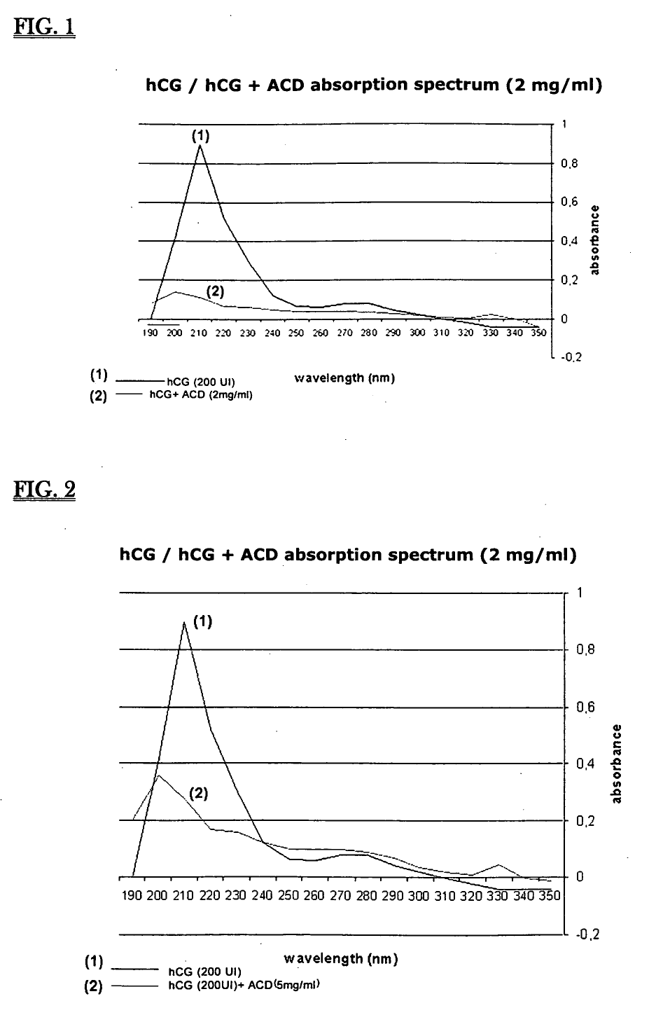

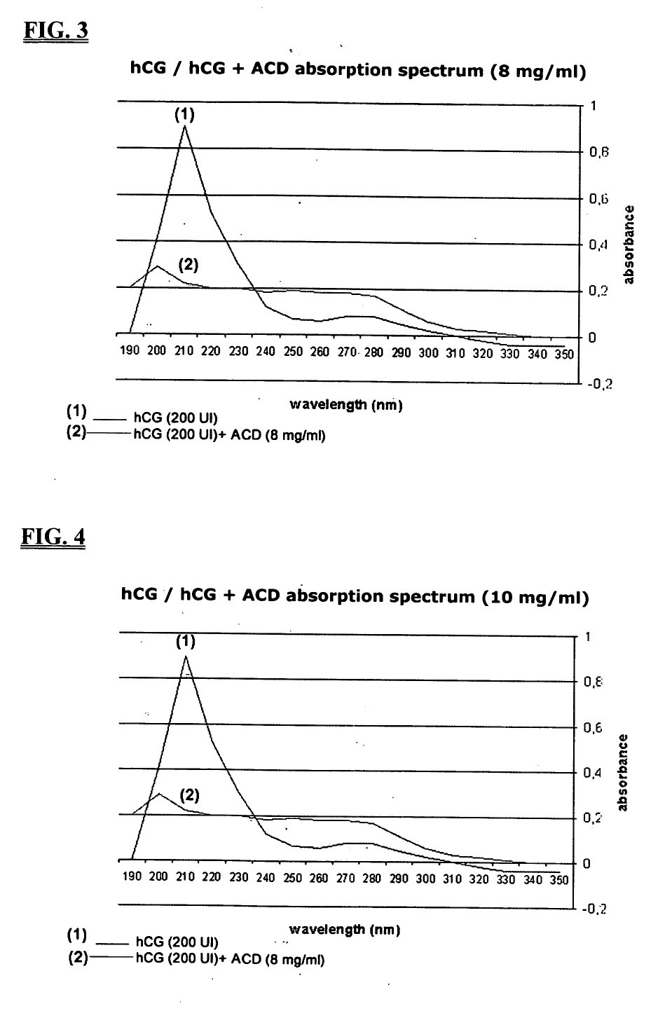

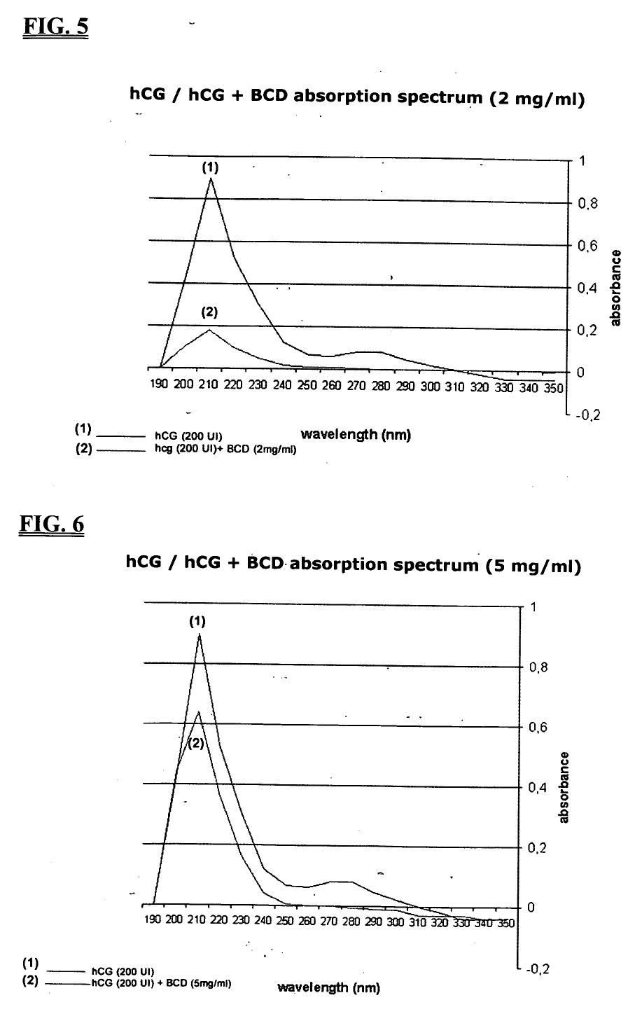

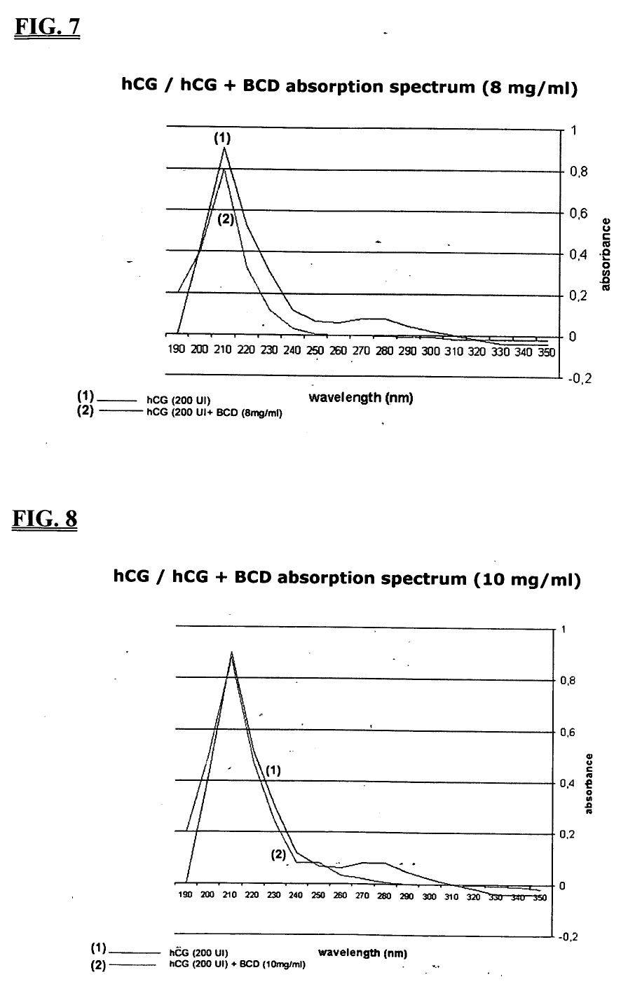

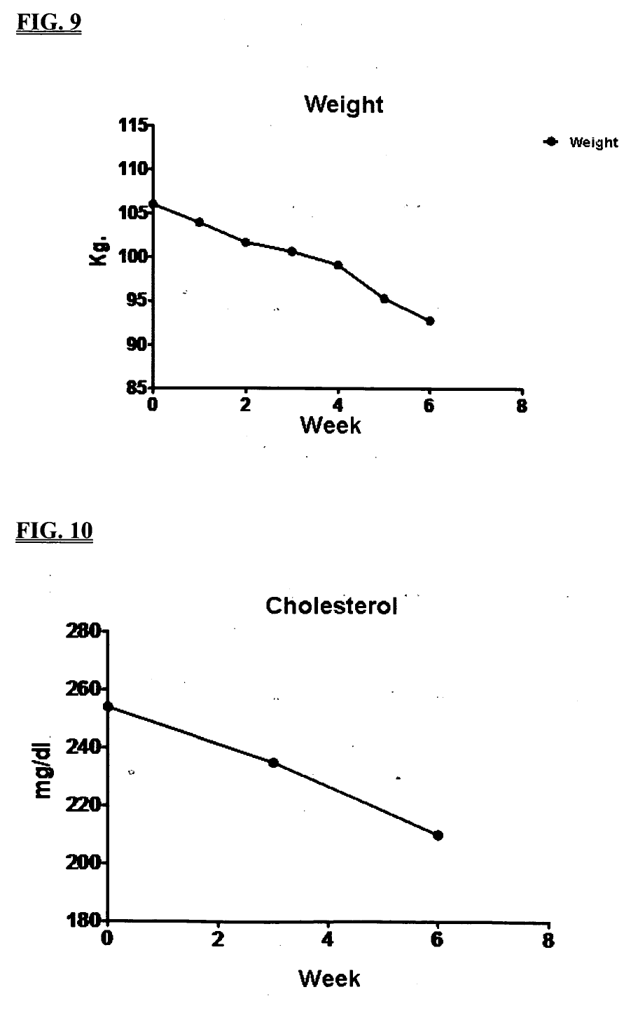

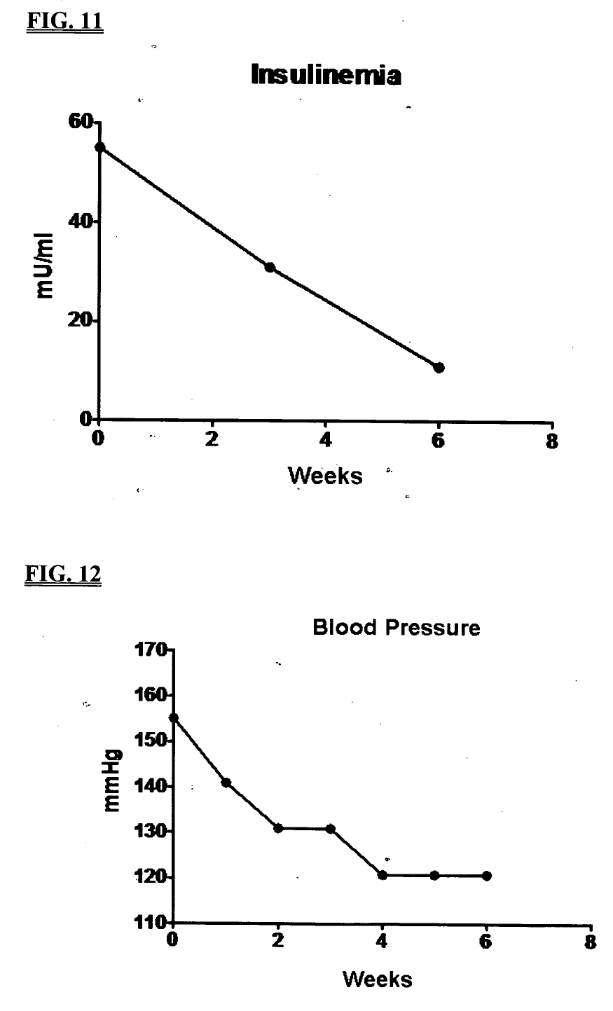

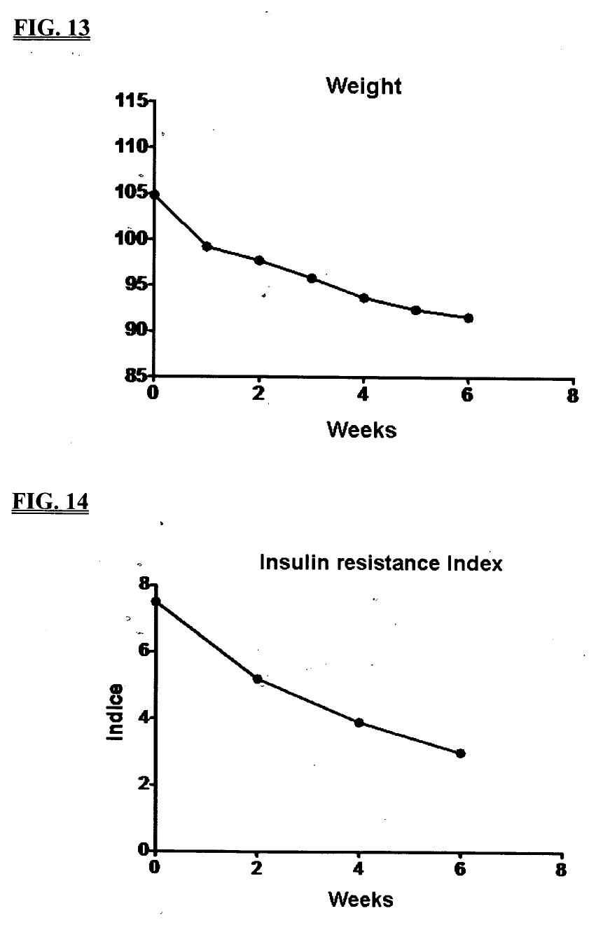

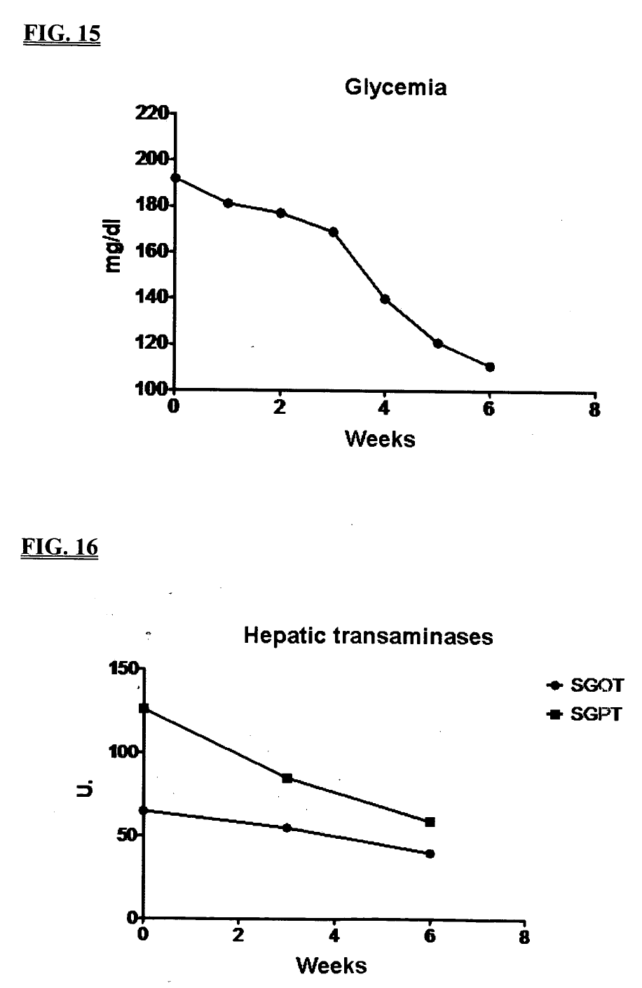

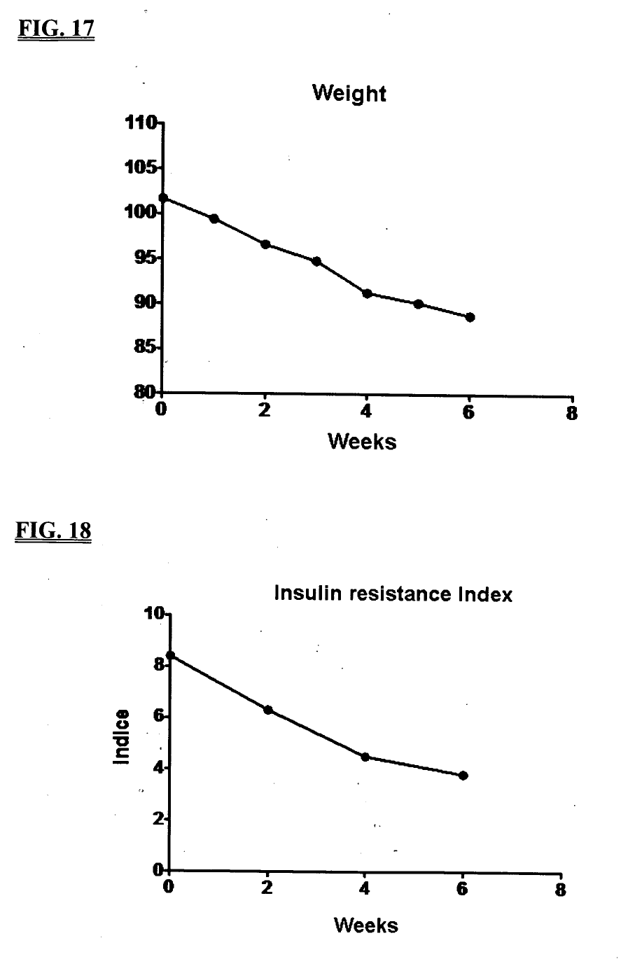

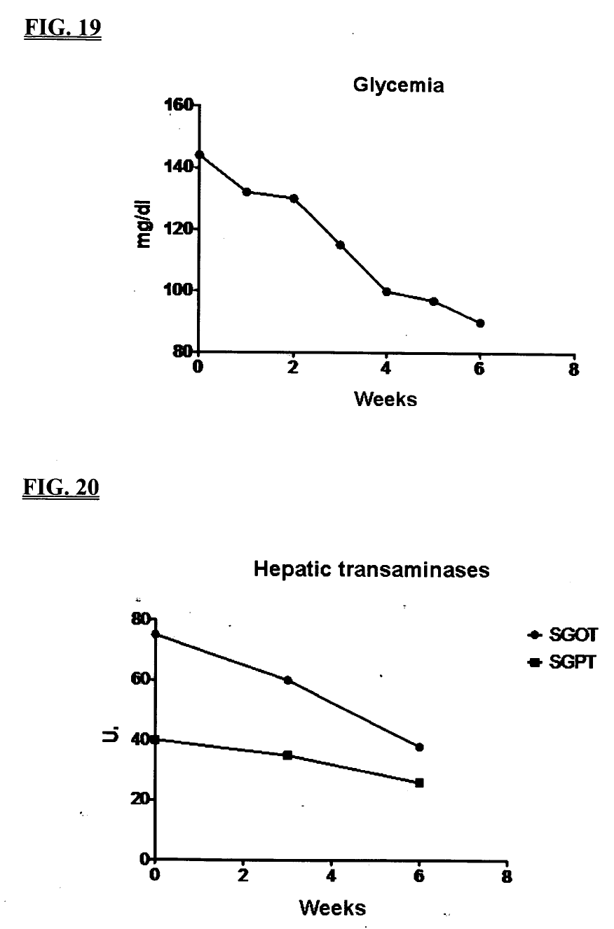

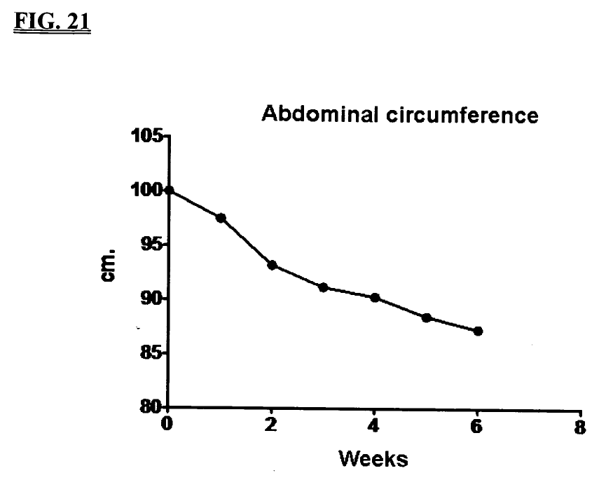

The present invention contemplates the use of cyclodextrins as carriers (transporters) and their capacity to form inclusion complexes with bioactive molecules (such as hCG), allowing the clinical activity of the hCG by mouth (oral), facilitating the administration of an originally prescribed medication in injectable form:

More specifically, the goal it is the creation of hCG (human Chorionic Gonadotropin)—cyclodextrins complexes, and their clinical use in different disorders from various pathologies, through use of oral pharmaceutical formulations.

### DETAILED DESCRIPTION OF INVENTION

This invention relates to the “Method to obtain the human Chorionic Gonadotropin (hCG)/cyclodextrin complex for oral administration, product obtained by this method and clinical and therapeutic use of the complex human Chorionic Gonadotropin (hCG)/cyclodextrin”.

### FIELD OF INTEREST

treatment of patients with arterial hypertension, overweight disorders, type 2 diabetes, or reactive hyperglycemia, hypertriglyceridemia and hypercholesterolemia.

### CURRENT TECHNIQUE

in traditional Pharmacopoeia the prescription of Chorionic Gonadotropin is indicated via intramuscular injection. The novelty of this invention is that facilitates the oral administration, avoiding all the disadvantages arising from the parenteral administration.

Human Chorionic Gonadotropin (hCG) is a glycoprotein encompassing the association between an α (alpha) and a β (beta) chains. The hCG is obtained from the urine of pregnant women and is not homogeneous.

Highly purified preparations of hCG also contain several fractions that differ in the sialic acid content and biological action. The potency of hCG is indicated in units of biological action.

The hormonal effect of Chorionic Gonadotropin is based on its capacity to stimulate the biosynthesis of sex steroids in the gonads (ovaries and testes). The hCG action is qualitatively the same as the one from the pituitary Gonadotropin (LH). However, hCG has a significantly longer half-life which leads to a stronger action in case of accumulative administration.

HCG stimulates in ovaries the granulosa, theca and stroma or luteal cells to maintain progesterone and estradiol production.

In granulosa cells from small follicles the biosynthesis of estradiol is preferentially stimulated by high doses of hCG. As in the granulosa cells of dominant mature follicles and/or luteinizing granulosa cells, progesterone biosynthesis of is stimulated by high doses of hCG. In addition, hCG stimulates the production of biologically active peptides in the ovary that are important for reproduction regulation (eg.: inhibition, relaxation, plasminogen-activator-inhibitor.

In Leydig cells hCG stimulates testosterone production and other sex steroids such as dihydrotestosterone, 17 OH-progesterone and estradiol.

While the primary indication of the hCG is related to the infertility area, our studies indicate that it can be successfully used in a extensive variety of diseases, without undesirable effects since it is a natural origin product.

### DESCRIPTION

Our invention is constituted by the formation of a cyclodextrin+Gonadotropin complex. The cyclodextrins are non-reducing oligosaccharides obtained by the enzymatic hydrolysis of starch.

Cyclodextrins are molecules that contain 6, 7 and 8 units of alpha-D-glucopyranose assembled in position 1-4 giving rise to cyclic structures called: Alpha cyclodextrin, Beta cyclodextrin and Gamma cyclodextrin.

These cyclical structures are constituted by ring-shaped rigid molecules with a central cavity, where primary and secondary hydroxyl groups of the glucopyranose units are oriented towards the outside area of the ring, conferring to the cyclodextrin molecule hydrophilic characteristics, while the central cavity lined up with carbon and ether-oxygen skeletal atoms residues from glucopyranose adopts lipophilic characteristics.

The characteristics of the central cavity allow the cyclodextrins to form inclusion complexes with biologically active molecules, in this case hCG.

The aim of this invention is to communicate a pharmaceutical formulation, whose composition includes hCG (human Chorionic Gonadotropin) stabilized with non-reducing oligosaccharides, specifically cyclic oligosaccharides known as cyclodextrins, allowing the formation of inclusion complexes, thus facilitating hCG absorption and metabolization through the oral route.

In the formation of these inclusion complexes the cyclical structure of cyclodextrin offers a central cavity with lipophilic characteristics, where molecules with an appropriated size or non-polar fractions of macromolecules can penetrate forming covalent unions and thus stabilizing the complex.

The formation of these inclusion complexes does not alter the characteristics of the molecule, either the complex or the host. Consequently the biologically active molecules retain their intrinsic capacity to permeabilise biologic membranes or interact with specific cell receptors.

In concordance with this concept, we propose the formation of an inclusion complex that we will identify as hCG*CD

hCG+CD′hCG*A/B/G−CD

Where:

hCG: human Chorionic Gonadotropin (glycoproteic hormone), represents the bioactive molecule capable of entering into the central cavity of the cyclodextrin.

(*) Represents the covalent unions between the bioactive molecule and cyclodextrin.

A/B/G−CD represents the cyclodextrin (alpha, beta or gamma cyclodextrins)

Materials and Methods: To prepare the complex the following materials have been used:
1. HCG 200 IU (human Chorionic Gonadotropin—lyophilized)
2. TACD-T (alpha cyclodextrin)—Trappsol (USA)
3. KLEPTOSE (beta cyclodextrin)—Roquete Freres (FRANCE)
4. Phosphoric acid 85%—Carlo Erba (ITALY)
5. Sodium Hydroxide (NaOH—Merck)
6. Water injectable quality (Roux Ocefa)
   Preparation of the complex: the cyclodextrin+hCG complex is prepared by dissolving the drug (hCG) in an aqueous solution of cyclodextrin with constant agitation under a specific temperature until an homogeneous solution is obtained.

The pH is adjusted to the desired value and finally the obtained solution is vacuum filtered with a 0.22 micron filter to maintain sterility.

HCG*CD complex solutions were prepared with the procedure described above and under laminar flow conditions and whose concentrations and details are described in tables 1 and 2.

TABLE 1

| Alpha cyclodextrin | NaCl | NaOH 0.1M or | hCG |
|---|---|---|---|
| (mg/ml) | (mg/ml) | H₃PO₄ 1:5 | (IU/ml) |
| 2 | 9 | cs ph 7 | 200 |
| 5 | 9 | cs ph 7 | 200 |
| 8 | 9 | cs ph 7 | 200 |
| 10 | 9 | cs ph 7 | 200 |

TABLE 2

| Beta cyclodextrin | Nacl | NaOH or H₃PO₄ | hCG |
|---|---|---|---|
| (mg/ml) | (mg/ml) | (mg/ml) | (IU/ml) |
| 2 | 9 | cs ph 7 | 200 |
| 5 | 9 | cs ph 7 | 200 |
| 8 | 9 | cs ph 7 | 200 |
| 10 | 9 | cs ph 7 | 200 |

All solutions described in Tables 1 and 2 have been analyzed by UV spectrophotometry scanning between 350 nm and 190 nm.

The spectrums were performed in a Beckman spectrophotometer, using quartz buckets with 10 mm thickness and maintaining its temperature at 22 degrees Celsius in thermostatic bath.

The obtained spectrums from the different studied solutions are displayed in the tables below.

As a guide and to facilitate the comparison it has been drawn together the absorption spectrum of a non-complexed hCG solution (200 IU/ml) in the same preparation conditions that the solutions under consideration.

Results: results detailed below were obtained in the formation of the hCG+cyclodextrins complexes, using different concentrations of alpha and beta-cyclodextrins (2 to 10 mg/ml), demonstrating that the formation of hCG/cyclodextrins complexes, such as described below, possess clinical and therapeutic utility.

These results are detailed in the graphics, in which:

GRAPHIC 1: shows the absorption spectrum of hCG (200 IU) and hCG (200 IU)+Alpha cyclodextrin (2 mg/ml).

GRAPHIC 2: shows the absorption spectrum of hCG (200 IU) and hCG (200 IU)+Alpha cyclodextrin (5 mg/ml).

GRAPHIC 3: shows the absorption spectrum of hCG (200 IU) and hCG (200 IU)+Alpha cyclodextrin (8 mg/ml).

GRAPHIC 4: shows the absorption spectrum of hCG (200 IU) and hCG (200 IU)+Alpha cyclodextrin (10 mg/ml).

GRAPHIC 5: shows the absorption spectrum of hCG (200 IU) and hCG (200 IU)+Beta cyclodextrin (2 mg/ml).

GRAPHIC 6: shows the absorption spectrum of hCG (200 IU) and hCG (200 IU)+Beta cyclodextrin (5 mg/ml).

GRAPHIC 7: shows the absorption spectrum of hCG (200 IU) and hCG (200 IU)+Beta cyclodextrin (8 mg/ml).

GRAPHIC 8: shows the absorption spectrum of hCG (200 IU) and hCG (200 IU)+Beta cyclodextrin (10 mg/ml).

GRAPHIC 9: shows in a coordinates chart the clinical case of a female patient, EC, 53 years old, with diagnosed hypercholesterolemia, hyperinsulinemia, arterial hypertension and overweight during a six-week treatment period, using the hCG+cyclodextrin complex by oral administration.

GRAPHIC 10: shows a coordinates chart in the clinical case of the same patient that the graphic above, showing the cholesterol evolution.

GRAPHIC 11: shows a coordinates chart in the clinical case of the same patient that graphic 9, indicating the evolution of insulinemia.

GRAPHIC 12: shows a coordinates chart in the clinical case of the same patient that graphic 9, indicating the evolution of blood pressure.

GRAPHIC 13: shows a chart of the case of a male patient, GB., Aged 30, diagnosed with a insulin resistance syndrome, fatty liver, non-insulin dependent diabetes and overweight, over a treatment period of 6 weeks, using the complex hCG+cyclodextrin by oral administration.

GRAPHIC 14: shows a chart of the case of the same patient in graphic 13, indicating the evolution of the insulin resistance index.

GRAPHIC 15: shows a chart of the case of the same patient in graphic 13, indicating the glycemia evolution.

GRAPHIC 16: shows a chart of the case of the same patient in graphic 13, indicating the evolution of the hepatic transaminases index.

GRAPHIC 17: shows a chart indicating the case of a female patient, AH., 54 years old, diagnosed with insulin resistance syndrome, hyperglycemia, abdominal obesity and fatty liver, for a period of 6 weeks of treatment, using the hCG+cyclodextrin complex by oral administration, indicating the evolution of body weight.

GRAPHIC 18: shows a chart of the case of the same patient in graphic 17, indicating the evolution of the insulin resistance index.

GRAPHIC 19: shows a chart of the case of the same patient in graphic 17, indicating the evolution of the glycemia index.

GRAPHIC 20: shows a chart of the case of the same patient in graphic 17, indicating the evolution of hepatic transaminases index.

GRAPHIC 21: shows the chart of the same patient in graphic 17 indicating the evolution of the abdominal circumference.

Conclusions: from the obtained spectrum results for the different formulations containing alpha and beta cyclodextrins it can be observed that they have different levels of complexes formation.

In the case of prepared solutions with alpha cyclodextrins we observed that the complexes formation degree is lower even with increasing concentration. This phenomenon could be explained bearing in mind that the size or area of the central cavity is small when compared to the size of the hCG (human Chorionic Gonadotropin) molecule.

We noticed that the solutions prepared with beta cyclodextrins have a greater tendency to complexes formation.

This behavior is noticed as long as the concentration of cyclodextrin is increased, but at the same time is limited, implying that there is an optimal concentration for the formation of an inclusion complex, which can be seen between 5 mg/ml and 8 mg/ml cyclodextrin.

The greater formation of complexes degree for the beta cyclodextrins is due to a larger volume or space in its central cavity that allows for greater inclusion of the molecule of hCG (human Chorionic Gonadotropin).

Therefore we concluded that the inclusion complexes hCG+CD′hCG*A/B/G−CD are feasible and that there is an optimal concentration which is in balance with complexed molecules or host, facilitating the stability and therapeutic activity of the formed complex.

The therapeutic activity of the hCG+cyclodextrin complex is evidenced by the modification of clinical parameters after its administration, demonstrated by the pertaining studies (see casuistry).

Problems with known treatments: for arterial hypertension, overweight disorders, type 2 diabetes or reactive hyperglycemias, hypertriglyceridemia or hypercholesterolemia clinical disorders, proposed treatments take into consideration different types of medications depending on the disease.

How this invention solves this problem: in our case the hCG+cyclodextrin complex is a unique medication that is offered as an alternative therapy. Furthermore, if We consider that currently only injectable pharmaceutical presentations of hCG are available, this invention possesses the following advantages:

1. Easy to administer.

2. Simplification of the medication election.

3. Possibility to administer hCG in an outpatient basis, at home, without having to attend a infirmary or health center to receive the intramuscular injection of the medication.

To summarize, the use of human Chorionic Gonadotropin complexed with cyclodextrins by oral administration presents undoubted advantages, providing the patient with the administration of medication by mouth and expanding its spectrum of clinical indications.

APPLICATION: Under medical supervision the patient is indicated to receive human Chorionic Gonadotropin complexed with cyclodextrin by oral route. Depending on the type of clinical indication, the daily doses of Gonadotropin are adjusted between 300 and 600 International Units per day (oral, retaining the solution of Gonadotropin for 1-2 minutes in the oral cavity to facilitate part of its absorption through the rich venous plexus of the oral mucosa, and then swallow) during the treatment period.

In cases of:
1. Arterial hypertension.
2. Overweight disorders.
3. Type 2 diabetes or reactive hyperglycemia.
4. Hypertriglyceridemia.
5. Hypercholesterolemia

Also indicated is a very low calories diet (about 500 calories/day), hypolipidic, hypohydrocarbonated, and proteins provided by 200 grams of animal protein, plus a combination of carbohydrates and vegetables to complete the indicated calories.

Those pathologies that do not require a dietary procedure only contemplate the hCG+cyclodextrin complex administration for their treatment.

The treatments are carried out for a period of not less than one month and can be extended up to three months. After that period a resting period of one month is indicated, after which the procedure can be repeated again.

How it works: the combined treatment of hCG+cyclodextrin and its action in:
1. Adipose tissue, inhibiting lipogenesis.
2. In the hypothalamic region, improving the concentration of neuropeptides (endorphins).
3. In the cardiovascular system, on arterial hypertension.
4. In the carbohydrate metabolism, non insulin dependant diabetes.
5. In the cortex-diencephalic region, inhibiting the irritative neural circuits, thus acting in behavioral disorders such as anxiety, neurosis, irritative states, stress, chronic fatigue syndrome.
6. Intermediary metabolism, lipids metabolism, triglycerides and cholesterol.

Clinical Cases Description:

Case 1. EC (Graphics 9, 10, 11 and 12)

Female patient. 53 years old. Diagnosis: hypercholesterolemia, hyperinsulinemia, arterial hypertension. Overweight.

Treatment period using the hCG+cyclodextrin complex: 5 weeks. Significant reduction of weight and the rest of her clinical and laboratory parameters.

Case 2. GB (Graphics 13, 14, 15 and 16)

Male patient. 30 years old. Diagnosis: insulin resistance syndrome, fatty liver, non-insulin dependent diabetes and overweight.

Significant improvement in his clinical and laboratory parameters after five treatment weeks with the hCG+cyclodextrin complex.

Case 3. AH (Graphics 17, 18, 19, 20 and 21).

1. 54 years old patient. Female. Diagnosis: insulin resistance syndrome. Hyperglycemia. Fatty liver. Abdominal obesity

Significant improvement in her clinical and laboratory parameters after 5 treatment weeks with the oral hCG+cyclodextrin complex.

What is described and represented in the attached graphics and charts clearly highlights the functional and constructive advantages that characterize this invention and is considered an important advance in this technology, to which experts in this area may add many more, asking to include this invention in the pertaining law on this matter, requesting this law for protection according to the claims that follow:

Having described and determined the nature and scope of the present invention and how it has to be implemented, we declare that what is claimed as exclusive property and invention is:

1) “METHOD FOR OBTAINING THE HUMAN CHORIONIC GONADOTROPIN (HCG)/CYCLODEXTRIN COMPLEX TO BE ORALLY ADMINISTERED” characterized by the preparation of the hCG+cyclodextrin complex in an aqueous solution of cyclodextrin maintaining constant agitation and a given temperature until obtaining an homogeneous solution, subsequently pH is adjusted to the desired value and finally the solution is vacuum-filtered with 0.22 micron filters in order to maintain its sterility; preparing afterwards, under laminar flow conditions, the complexes' solutions.

2) “PRODUCT OBTAINED WITH THE HUMAN CHORIONIC GONADOTROPIN (HCG)/CYCLODEXTRIN METHOD” 1), characterized by a solution of hCG*CD complex whose concentrations and details are indicated in the subsequent tables 1 and 2:
TABLE 1

| Alfa cyclodextrin | NaCl | NaOH 0.1M or | hCG |
|---|---|---|---|
| (mg/ml) | (mg/ml) | H₃PO₄ 1:5 | (IU/ml) |
| 2 | 9 | cs ph 7 | 200 |
| 5 | 9 | cs ph 7 | 200 |
| 8 | 9 | cs ph 7 | 200 |
| 10 | 9 | cs ph 7 | 200 |

TABLE 2

| Beta cyclodextrin | NaCl | NaOH or H₃PO₄ | hCG |
|---|---|---|---|
| (mg/ml) | (mg/ml) | (mg/ml) | (IU/ml) |
| 2 | 9 | cs ph 7 | 200 |
| 5 | 9 | cs ph 7 | 200 |
| 8 | 9 | cs ph 7 | 200 |
| 10 | 9 | cs ph 7 | 200 |

3) “METHOD FOR OBTAINING THE HUMAN CHORIONIC GONADOTROPIN/CYCLODEXTRIN COMPLEX TO ADMINISTER BY ORAL ROUTE, PRODUCT OBTAINED BY THIS METHOD AND CLINICAL AND THERAPEUTICAL USE OF THE HUMAN CHORIONIC GONADOTROPIN (HCG)/CYCLODEXTRIN COMPLEX” as claimed in any of the previous claims characterized by being implemented under medical supervision, indicating the patient human Chorionic Gonadotropin (hCG) by oral administration, complexed with cyclodextrin, depending on the type of clinical indication the daily doses of Gonadotropin are adjusted from 300 to 600 International Units, oral, retaining the solution of gonadotropin for 1-2 minutes in the mouth before swallowing, to be administered during the treatment period.
